(19) United States
(12) Patent Application Publication
(10) Pub. No.: US 2025/0276149 A1
(43) Pub. Date: Sep. 4, 2025

(54) HYBRID OXYGEN DELIVERY SYSTEM AND PROCESS

(71) Applicant: Seabeck Holdings, LLC, Seabeck, WA (US)

(72) Inventor: James J. Ruttler, Seabeck, WA (US)

(73) Assignee: Seabeck Holdings, LLC, Seabeck, WA (US)

(21) Appl. No.: 19/069,074

(22) Filed: Mar. 3, 2025

Related U.S. Application Data
(60) Provisional application No. 63/560,752, filed on Mar. 3, 2024.

(51) Int. Cl. A61M 16/10; A61M 16/00

(52) U.S. Cl. CPC A61M 16/101; A61M 16/0093; A61M 16/1015; A61M 16/107; A61M 2016/0027; A61M 2016/003; A61M 2016/1025; A61M 2202/0208

(57) ABSTRACT

In one embodiment, a hybrid oxygen delivery system includes, but is not limited to, a pressurized oxygen vessel configured to store a first source of oxygen under pressure; an oxygen concentrator configured to generate a second source of oxygen from ambient air; a flow control valve configured to combine the first source of oxygen with the second source of oxygen for delivery to one or more stations; plumbing that fluidly connects the pressurized oxygen vessel and the oxygen concentrator to the flow control valve; and a processor configured to actuate the flow control valve based on one or more parameters to deliver oxygen to one or more stations.

### PRIORITY CLAIM

The present application is a non-provisional of and claims the benefit of and/or priority to the following application(s) under 35 USC 119 and/or 120: U.S. Provisional Patent Application 63/560,752 filed Mar. 3, 2024 (docket JIM-P-48). The foregoing application(s) are incorporated by reference in their entirety as if fully set forth herein.

### FIELD OF THE INVENTION

This invention relates generally to oxygen system technology, and more specifically, to a hybrid oxygen delivery system and process.

### SUMMARY

This invention relates generally to oxygen system technology, and more specifically, to a hybrid oxygen delivery system and process.

In one embodiment, a hybrid oxygen delivery system includes, but is not limited to, a pressurized oxygen vessel configured to store a first source of oxygen under pressure; an oxygen concentrator configured to generate a second source of oxygen from ambient air; a flow control valve configured to combine the first source of oxygen with the second source of oxygen for delivery to one or more stations; plumbing that fluidly connects the pressurized oxygen vessel and the oxygen concentrator to the flow control valve; and a processor configured to actuate the flow control valve based on one or more parameters to deliver oxygen to one or more stations.

In one embodiment, an oxygen concentrator system includes, but is not limited to, an oxygen concentrator configured to generate oxygen from ambient air; an oxygen reservoir configured to store the oxygen in reserve when excess oxygen is generated; a recapture system configured to source oxygen from the oxygen reservoir and the concentrator when a shortage of oxygen is generated; plumbing that fluidly connects the oxygen concentrator, the oxygen reservoir, and the recapture system; and a processor configured to control the recapture system based on one or more parameters to deliver oxygen to one or more stations.

In one embodiment, a hybrid pulse demand oxygen system includes, but is not limited to, an oxygen concentrator configured to generate oxygen at a first rate; an oxygen reservoir configured to store the oxygen generated at the first rate; an oxygen pressure vessel configured to store pressurized reserve oxygen; a re-pressurization pump configured to increase pressure of the oxygen stored in the oxygen reservoir; a pulse demand system configured to deliver oxygen at a variable rate based on one or more parameters; plumbing configured to fluidly connect the oxygen concentrator, the oxygen reservoir, the oxygen pressure vessel, the re-pressurization pump, and the pulse demand system; and a processor configured to variably source oxygen from the re-pressurization pump and the oxygen pressure vessel based on a volume of oxygen available in the oxygen reservoir.

In one embodiment, a process for producing hybrid oxygen flow includes, but is not limited to, obtaining intake ambient air; compressing the ambient intake air; filtering nitrogen from the ambient intake air to concentrate oxygen using pressure swing adsorption; measuring one or more physiological parameters; detecting that the one or more physiological parameters has crossed one or more thresholds; supplementing the oxygen with reserve oxygen in response to detecting that the one or more physiological parameters has crossed the one or more thresholds; measuring a flow of the oxygen; and outputting the oxygen to one or more breathing stations.

In one embodiment, a process for recapturing concentrated oxygen flow includes, but is not limited to, obtaining intake ambient air; compressing the ambient intake air; filtering nitrogen from the ambient intake air to concentrate oxygen using pressure swing adsorption; filling a reservoir with the oxygen continuously; measuring one or more physiological parameters; detecting that the one or more physiological parameters has crossed one or more thresholds; pumping oxygen from the reservoir at a rate that is based on the one or more physiological parameters; measuring a flow of the oxygen; and outputting the oxygen to one or more breathing stations.

In one embodiment, a process for pulsing concentrated oxygen includes, but is not limited to, obtaining intake ambient air; compressing the ambient intake air; filtering nitrogen from the ambient intake air to concentrate oxygen using pressure swing adsorption; filling a reservoir with the oxygen continuously; re-pressurizing a portion of the oxygen in the reservoir to create a standby charge for at least one pulse dosage; measuring one or more physiological parameters; detecting that the one or more physiological parameters has crossed one or more thresholds; pulsing oxygen using the standby charge in response to detected breathing in an amount that is based on the one or more physiological parameters; measuring a flow of the oxygen; and outputting the oxygen to one or more breathing stations.

Additional details may be included in any of these embodiments as illustrated, discussed, or claimed herein.

### BRIEF DESCRIPTION OF DRAWINGS

Embodiments of the present invention are described in detail below with references to the following drawings

FIG. 1 is a system diagram of a hybrid oxygen delivery system, in accordance with an embodiment of the invention;

FIG. 2 is a system diagram of a hybrid oxygen delivery system, in accordance with an embodiment of the invention;

FIG. 3 is a process diagram of a hybrid oxygen delivery system, in accordance with an embodiment of the invention;

FIG. 4 is a system diagram of a hybrid oxygen delivery system, in accordance with an embodiment of the invention;

FIG. 5 is a system diagram of a hybrid oxygen delivery system, in accordance with an embodiment of the invention;

FIG. 6 is a process diagram of a hybrid oxygen delivery system, in accordance with an embodiment of the invention;

FIG. 7 is a system diagram of a hybrid oxygen delivery system, in accordance with an embodiment of the invention;

FIG. 8 is a system diagram of a hybrid oxygen delivery system, in accordance with an embodiment of the invention;

FIG. 9 is a process diagram of a hybrid oxygen delivery system, in accordance with an embodiment of the invention; and

APPENDIX A is a manual for a hybrid oxygen delivery system and process, in accordance with an embodiment of the invention. APPENDIX A is incorporated by reference in its entirety as if fully set forth herein.

### DETAILED DESCRIPTION

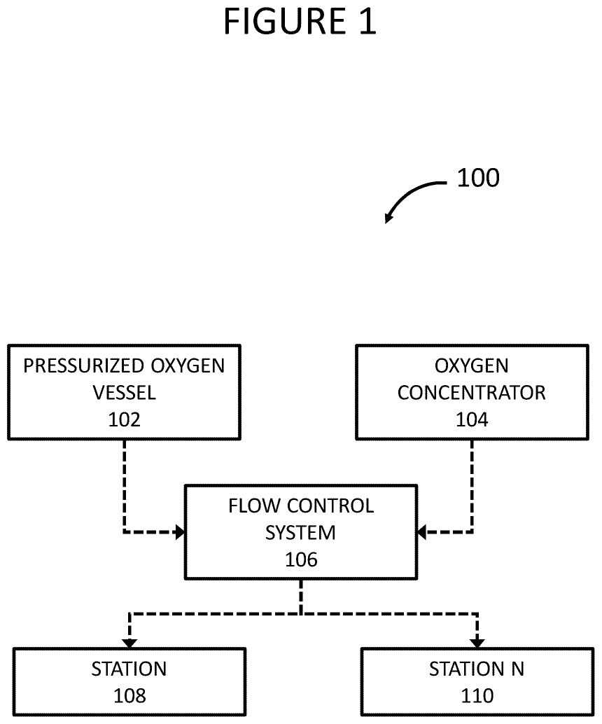

This invention relates generally to oxygen system technology, and more specifically, to a hybrid oxygen delivery system and process. Specific details of certain embodiments of the invention are set forth in the following description and in FIGS. 1-9 and APPENDIX A to provide a thorough understanding of such embodiments. The present invention may have additional embodiments, may be practiced without one or more of the details described for any described embodiment, or may have any detail described for one embodiment practiced with any other detail described for another embodiment.

In one embodiment, a hybrid oxygen delivery system and process are optimized for aviation. In this embodiment, the system generates oxygen using 80 W of electrical power and only dips into a pressure oxygen bottle supply when needed, such as at high altitudes or on user request. When the concentrated oxygen supply is sufficient for the mission, the bottled oxygen supply remains unused. Thus, the system is a hybrid system that combines a concentrator and an oxygen bottle. The concentrator provides virtually unlimited oxygen (e.g., 1 LPM to support one person up to 18 k MSL and two people up to 15 k MSL), without use of the oxygen bottle. The oxygen bottle is on standby to further increase the oxygen flow (e.g., above 15 k MSL), or as needed, for an additional oxygen supply (e.g., 0.5-2.5 LPM). The bottle oxygen endurance depends largely on the altitude, bottle size, and number of users. With flights below the altitude thresholds (e.g., 18 k for one or 15 k for two), the bottled oxygen endurance is nearly infinite as the concentrator supplies all the needed oxygen using PSA oxygen concentration technology. With the oxygen injection mode activated, the provided oxygen bottle supplies an additional flow (e.g., 0.5 LPM-1 LPM). Further, the oxygen bottle also serves as a backup supply of oxygen independent of the system. A separate backup plumbing line and emergency cannula plug are included to make oxygen available even in the event of electrical failure or system malfunction. The system may include the Health View avionics system. Along with output of total flow rate, oxygen injection flow rate, oxygen bottle pressure, and onboard temperature, the Healthview controls the injection with a control that regulates the oxygen injection. Usable with the system are Illyrian II oximeter, Aithre Boom Cannulas, Aithre nasal pillow mask, and the Aithre Shield EX 3.0 carbon monoxide detector.

In one embodiment, the concentrator system concentrates oxygen real-time, such as during flight, anytime, anywhere, using a fraction of the capacity of standard electrical alternators. Based on proven pressure swing adsorption technology (PSA), the concentrator outputs continuous >93% pure oxygen at (e.g., at 1 LPM) using just 6 amps or 80 watts with 12-14V aircraft power. A compressor pump is used to pressurize intake air and feed it through zeolite sieve beds. The sieve beds retain the nitrogen in the air and pass oxygen through the adsorption technique (PSA). The concentrator is capable of operating above 10 k MSL where the air is thinner.

In one embodiment, any pressure oxygen cylinder or bottle may be used with the system, such as the Aithre 47 L composite bottle. For instance, this bottle is constructed from lightweight composite carbon fiber with an aluminum lining and offers a 2000 PSI working pressure and includes a built-in backup adjustable flow regulator. The Aithre 47 L bottle includes a standard CGA-540 fill port, but it is one-way and doesn't require removal of any parts to fill. A metal handle provides enhanced protection against impact. In this example, there are two output ports on the regulator. The first output port is used for backup and allows for continuous flow settings (e.g., adjustable to 0.5-15 LPM). The second output port is an always-on port that connects to the hybrid oxygen system. Further, the Aithre 47 L bottle includes the standard analog pressure gauge, and the Aithre Meso wireless pressure gauge. This provides 0-2500 PSI readings to the Healthview and to the Aithre Connect IOS app in increments of 10 PSI to permit remote pressure monitoring.

In certain embodiments, unlike medical portable oxygen concentrators, the system does not use a Lithium battery. Instead, the system relies exclusively on ship power 12-14V, using the aircraft battery or engine-driven electrical alternator. At just 80 W or approximately 6 amps at a 14V supply, the system can fit within virtually any alternator capacity, and this results in reduced maintenance, a longer service life, and increased flight safety.

In one embodiment, the system is defined by a small footprint of 22.5 cm×14.5 cm×15 cm and a weight of just 6.8 lbs., plus the bottle size at 6.5 cm×33 cm overall and 3 lbs. filled. Thus, the system is small enough and light enough to free up space and expand useful load as compared to traditional legacy bottle systems.

For examples, where the system is installed in an aircraft, the system features panel-mounted cannula plugs, remote push-button activation, hidden built-in plumbing, and the Healthview avionics system. A unique hook mount system is provided for removably securing the system to a wall, floor, or supporting member. While the system should have intake access to fresh air through its ports, with center of gravity impact minimized, there are many options for mounting: tail-cone, nosecone (for pusher or twin-engine aircraft), or the baggage compartment. A rubber quick release mount is provided to removably secure the 47 L oxygen bottle in the baggage compartment or in another accessible location. With quick release bulkhead fittings, the 47 L bottle can be easily removed for transfilling and reconnected to a remotely located concentrator hybrid system.

In one embodiment, the system features automatic digital flow control of concentrated oxygen. The flow is set and managed automatically to help support flight safety with predictable and optimized oxygen generation. Simply push the oxygen button and the oxygen is provided to (e.g., 1 LPM at altitudes up to 18 k MSL and >93% concentration). Oxygen injection is default off, unless requested in the Aithre Connect app or the Healthview. The request in the Healthview is made via a mode soft button switch, which begins oxygen injection (e.g., above 15 k MSL at 0.5 LPM with increases to 1 LPM at 18 k MSL) for a combined concentrator-injection flow (e.g., 2 LPM at 18 k MSL). The Aithre Connect iOS app provides enhanced control over oxygen injection. In addition to mode switch, the Aithre Connect app enables injection at all altitudes with the ON mode. Also using the Aithre Connect app, the dosing algorithm can be scaled from 5% to 200% to fine-tune control of the oxygen injection. The Aithre Healthview II features the functionality in the Aithre Connect app.

In another embodiment, the system constantly monitors total flow, onboard temperature, pressure altitude, oxygen injection flow, bottle oxygen pressure, and faults. Data is output via serial to the Aithre Healthview and via BLE to the Aithre Connect iOS app. For supporting avionics, a discrete output 0-12V may be connected to throw a display message in response to a fault condition.

In one embodiment, the system includes the Healthview to both control oxygen delivery and output health information, including gauges for blood oxygen, heart rate, respiration rate, flow rate, cabin pressure, cabin temperature, carbon monoxide, and oxygen tank pressure. The Healthview features a display with rich and vibrant gauges, enclosed in an ultra-compact aluminum case. The Healthview provides a low heat, low power, high functionality display, using only 150 mA of current at 14V. The Healthview works as a standalone device for cabin pressure altitude, using an internal barometric pressure sensor. Additionally, the Healthview dynamically expands to feature any available Aithre device, wirelessly and with or without use of the popular Aithre Connect iOS app. The Healthview is compatible with all Aithre devices, including the Shield series of carbon monoxide detectors, the Illyrian oximeter, the Meso oxygen pressure, the Metis wingtip temperature/dewpoint sensor, and the AVI and Turbo oxygen systems. In one specific example, power requirements are 12-18V and only 150 mA (0.15 A) and dimensions are 90 mm×61 mm×21.5 mm and weight is just under 6 ounces.

In a further embodiment, the system includes the Aithre Altus Meso wireless tank pressure gauge, which allows for monitoring the oxygen pressure remaining while the bottle is out of sight. Readings from 0-2500 PSI are transmitted to the Healthview and the Aithre Connect iOS app to supplement the analog pressure gauge.

Additionally, the system can include an Illyrian II oximeter which provides continuous real-time SPO2 and heart rate information to the Healthview and to the Aithre Connect iOS app. Advanced features include head movement and cabin altitude monitoring with haptic vibration feedback.

While the system can be used with a cannula, the system can optionally be used with an Aithre Boom Cannula to make oxygen delivery safer and more comfortable. The Boom Cannula features a low-profile aluminum anodized swivel adapter and an articulating boom arm, make oxygen delivery as easy and convenient as talking on the microphone. When not in use, the Boom Arm can be pushed or swiveled up and out of the way and can be easily recharged with a new nasal arm after extended periods of usc.

Optionally, the Turbo O2i system can include the Shield EX 3.0. The Shield CO detectors offer superior 10 year no recalibration service with sensitivity below 10 ppm and responsiveness of under 60 seconds. And the Shield EX 3.0 outputs CO, SPO2, and O2 PSI to the 210 Garmin, Dynon, and Advanced systems for additional safety monitoring.

In one embodiment, the oxygen concentration produced by the system is >93% for 2000 hours or 2 years, whichever comes first. There are different methods to verify the concentration and ensure that the system is operating correctly, using the Aithre Illyrian II wearable oximeter, using a portable oxygen concentration tester, or using a built-in concentration sensor. Continuously measured blood oxygen provides a direct indication of the sufficiency of breathing oxygen during flight. The Aithre Illyrian II provides a continuous readout of blood oxygen to modern avionics, the Aithre Healthview, the Aithre Connect iOS app, and via native haptic vibratory alerts.

There are several significant safety benefits of the system. Notably, the system does not need to store as much pressurized oxygen, so the risks associated with carrying, transporting, and filling pressurized oxygen vessels can be reduced using a smaller oxygen bottle. Additionally, the system does not require a Lithium battery, which helps mitigate battery fire risk.

Other safety features of the system optionally include high temperature Tefzel internal wiring, multiple differently oriented high speed cooling fans, over-temperature monitoring with auto shutdown, and capacitive electrical noise reduction. 225

FIG. 1 is a system diagram of a hybrid oxygen delivery system, in accordance with an embodiment of the invention. The system 100 includes a pressurized oxygen vessel 102, an oxygen concentrator 104, a flow control system 106, station 108, and one or more optional additional stations 110. The flow control system 106 controls the amount of oxygen sourced from either the pressurized oxygen vessel 102 or the oxygen concentrator 104. The oxygen concentrator 104 provides virtually unlimited oxygen (e.g. 2000-3000 hours) but has volume limits that can be insufficient at high density altitudes or for multiple stations. Furthermore, the oxygen concentrator 104 could malfunction or lose access to power. For any of these reasons, the pressurized oxygen vessel 102 can serve to supplement or as a backup to oxygen generated by the oxygen concentrator 104. The flow control system 106 can blend the oxygen in variable amounts from 0-100% sourced from the pressurized vessel 102.

In an aircraft environment, the system 100 is installed such that the oxygen concentrator 104 is hooked up to aircraft power and provides oxygen output to the stations 108 and 110 via the flow control system 106. The pressurized oxygen vessel 102 is fluidly connected to the flow control system 106, to enable the flow control system 106 to blend the oxygen delivered to the stations 108 and 110. Typically, the flow control system 106 defaults to 100% oxygen sourced from the oxygen concentrator 104. As the aircraft climbs altitude, the flow control system 106 supplements the oxygen from the oxygen concentrator 104 with increasing amounts of oxygen sourced from the pressurized oxygen vessel 102. For example, the oxygen concentrator 104 outputs 1 LPM continuously, with oxygen from the pressurized vessel 102 providing between 0.1 LPM-2.5 LPM linearly beginning above 15 k MSL up to 25 k MSL. Other volumes and ratios are contemplated depending upon the mission needs and the available space, weight, and alternator or battery capacity.

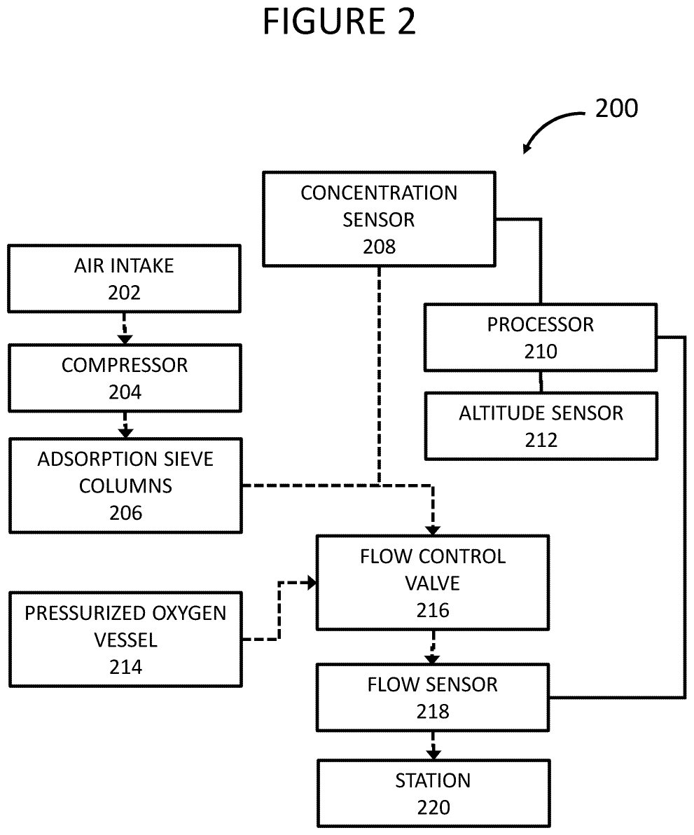

FIG. 2 is a system diagram of a hybrid oxygen delivery system, in accordance with an embodiment of the invention. In one embodiment, a hybrid oxygen delivery system 200 includes, but is not limited to, a pressurized oxygen vessel 214 configured to store a first source of oxygen under pressure; an oxygen concentrator including an air intake 202, compressor 204, and adsorption sieve columns 206 configured to generate a second source of oxygen from ambient air; a flow control valve 216 configured to combine the first source of oxygen with the second source of oxygen for delivery to one or more stations 220; plumbing, indicated by dashed lines, that fluidly connects the pressurized oxygen vessel and the oxygen concentrator to the flow control valve 216; and a processor 210 configured to actuate the flow control valve 216 based on one or more parameters to deliver oxygen to one or more stations 220. In one embodiment, processor 210 is electrically connected with oxygen concentration sensor 208, an altitude sensor 212, or a flow sensor 218. In one embodiment, the processor 210 is configured to actuate the flow control valve 216 based on one or more parameters including concentration, altitude, physiological demand, or flow.

Oxygen concentrators are devices that provide supplemental oxygen by filtering and concentrating oxygen from the surrounding air. The concentrator takes in ambient air, which contains about 21% oxygen, 78% nitrogen, and 1% other gases. The air passes through filters to remove dust and other impurities. The filtered air is then compressed. The compressed air moves through a molecular sieve, which uses materials like zeolite to adsorb nitrogen, leaving behind a higher concentration of oxygen. The concentrated oxygen (up to 99% purity) is then delivered to the user through a nasal cannula or mask.

The most common materials used for molecular sieves in oxygen concentrators are zeolites. These are microporous, aluminosilicate minerals that are highly effective at adsorbing nitrogen due to their unique crystalline structure. Carbon Molecular Sieves are usable in some applications for their ability to separate gases based on molecular size. However, they are less common in oxygen concentrators compared to zeolites. Silica gel can be used in gas separation processes. However, zeolites are most common because of their high efficiency, durability, and ability to selectively adsorb nitrogen while allowing oxygen to pass through.

An oxygen pressurized vessel, commonly known as an oxygen tank, is a storage container designed to hold oxygen under high pressure. These vessels come in two main types. High-pressure oxygen cylinders are typically made of steel, composite, carbon fiber or aluminum and are used to store oxygen gas at pressures up to 3,000 psi (20 MPa). They are commonly used in medical settings, aviation, scuba diving, and industrial applications. Cryogenic storage tanks store liquid oxygen at very low temperatures. They are used in aviation, medical facilities, industrial processes, and for rockets.

Flow control valves can include various electromechanical devices. Solenoid valves are electromechanical devices used to control the flow of liquids or gases. Here's an explanation of how they work. A solenoid coil is the core component is the solenoid coil, which is an electric coil with a ferromagnetic core (plunger) inside it. When an electric current passes through the coil, it generates an electromagnetic field. This magnetic field moves the plunger either up or down, depending on the valve design. The movement of the plunger opens or closes the valve, thereby controlling the flow of the fluid. There are different types of solenoid valves, including 2-way and 3-way, and NC or NO. For Normally Closed (NC), the valve remains closed when the solenoid is not energized. When the coil is energized, the plunger lifts, opening the valve and allowing fluid to pass through. For Normally Open (NO), the valve remains open when the solenoid is not energized. When the coil is energized, the plunger moves to close the valve, stopping the flow of fluid.

Other flow control valves are also included within the scope of the disclosure. Ball valves use a spherical plug with a hole through the middle. When the hole aligns with the flow, the valve is open; when it's perpendicular, the valve is closed. They are known for their durability and excellent shut-off capabilities. Gate valves are used primarily for on/off control. They operate by lifting a gate out of the path of the fluid. When fully open, gate valves offer minimal resistance to flow. Globe valves have a movable disk-type clement and a stationary ring seat in a generally spherical body. They work well for throttling and regulating flow. Butterfly valves use a rotating disk to control the flow. When the disk is turned parallel to the flow, the valve is open; when perpendicular, it is closed. They are lightweight and provide quick shut-off. Needle valves offer precise control of flow. They have a small port and a threaded, needle-like plunger that allows for fine adjustments. Check valves allow fluid to flow in one direction only, preventing backflow. They open with forward flow and close against reverse flow.

Oxygen concentration sensors, also known as oxygen sensors, measure the proportion of oxygen in a gas or liquid. They operate using various technologies, such as electrochemical, zirconia, and optical methods. In electrochemical sensors, a chemical reaction generates an electrical current proportional to the oxygen level. Zirconia sensors use a ceramic element that produces a voltage when exposed to oxygen, while optical sensors detect changes in light intensity caused by oxygen presence. These sensors ensure accurate and reliable oxygen measurements.

Altitude sensors, also known as altimeters, measure the height of an object above a reference level, typically sea level. They operate primarily by detecting changes in atmospheric pressure, which decreases predictably with increasing altitude. Mechanical altimeters use aneroid barometers to sense pressure changes and convert them into altitude readings through mechanical linkages. Modern electronic altimeters use precise sensors and microprocessors to provide accurate altitude data, often compensating for environmental factors like temperature and humidity. Additionally, specialized altimeters, such as radar and GPS-based altimeters, use radio waves and satellite signals, respectively, to determine altitude, making them essential in aviation, mountaineering, and various scientific applications.

Gas flow sensors measure the flow rate of gases by detecting changes in physical properties such as pressure, temperature, or velocity. Common types include differential pressure sensors, which measure the pressure drop across a constriction to calculate flow rate, and thermal mass flow sensors, which use the cooling effect of gas flow on a heated element to determine flow rate. These sensors convert the detected changes into electrical signals, providing accurate and real-time flow measurements. Gas flow sensors ensure efficient and safe gas management.

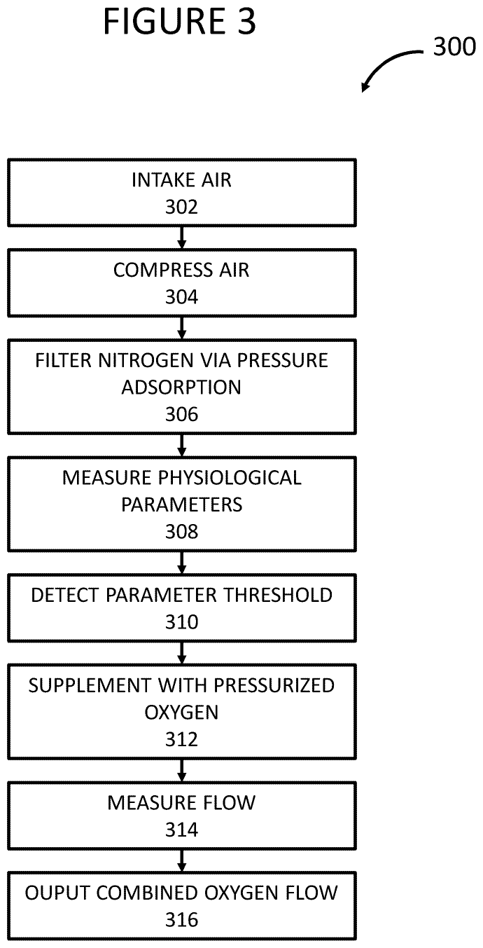

FIG. 3 is a process diagram of a hybrid oxygen delivery system, in accordance with an embodiment of the invention.

In one embodiment, a process 300 for producing hybrid oxygen flow includes, but is not limited to, obtaining intake ambient air at 302; compressing the ambient intake air at 304; filtering nitrogen from the ambient intake air to concentrate oxygen using pressure swing adsorption at 306; measuring one or more physiological parameters at 308; detecting that the one or more physiological parameters has crossed one or more thresholds at 310; supplementing the oxygen with reserve oxygen in response to detecting that the one or more physiological parameters has crossed the one or more thresholds 312; measuring a flow of the oxygen at 314; and outputting the oxygen to one or more breathing stations at 316. The process 300 can implement any of the 340 embodiments disclosed herein with respect to various systems.

SpO2 (peripheral oxygen saturation) and heart rate are critical indicators of a person's oxygenation status and overall cardiovascular health. SpO2 measures the percentage of oxygen-saturated hemoglobin in the blood, typically using a pulse oximeter. Normal SpO2 levels range from 95% to 100%, indicating sufficient oxygen supply to the body's tissues. Heart rate, measured in beats per minute, reflects the number of times the heart contracts to pump blood. Together, these metrics help assess the effectiveness of supplement oxygen. Monitoring SpO2 and heart rate ensures that persons receive the appropriate amount of supplemental oxygen, preventing hypoxia (low oxygen levels) and maintaining optimal cognitive function.

Cognitive functioning can be significantly impaired when SpO2 (peripheral oxygen saturation) levels drop below 90%. At this threshold, the brain receives insufficient oxygen, leading to symptoms such as confusion, difficulty concentrating, memory problems, and impaired judgment. Prolonged exposure to low oxygen levels, known as hypoxia, can exacerbate these cognitive issues and potentially cause long-term neurological damage. Maintaining SpO2 levels within the normal range of 95-100% is important for optimal brain function and overall health. Thus, the supplementation with pressurized oxygen at 312 can begin when SpO2 drops below 90% or heart rate exceeds 100 BPM, for example.

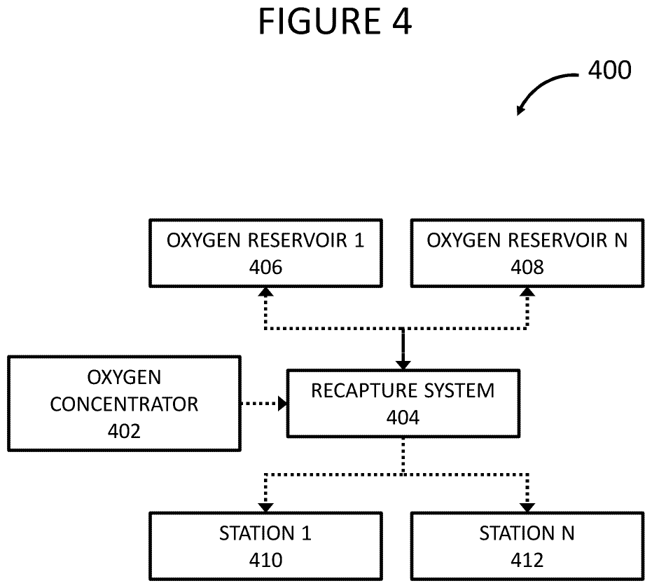

FIG. 4 is a system diagram of a hybrid oxygen delivery system, in accordance with an embodiment of the invention.

In one embodiment, system 400 includes an oxygen concentrator 402, a recapture system 404, an oxygen reservoir 406, one or more optional additional reservoirs 408, station 410, and one or more optional additional stations 412. The oxygen concentrator 402 operates continuously and fills the oxygen reservoir 406. When the demand from station 410 for oxygen is lower than the production rate of the oxygen concentrator 402, the oxygen reservoir 406 is filled with oxygen. However, when the demand from station 410 for oxygen is greater than the production rate of the oxygen concentrator 402, the recapture system 404 supplements the output from the oxygen concentrator 402 with recaptured oxygen from the oxygen reservoir 406. For instance, in an aircraft environment the oxygen concentrator 402 operates on the ground and at lower altitudes when the station 410 does not demand oxygen. The excess oxygen is stored in the oxygen reservoir 406. As the aircraft climbs in altitude, the station 410 demands more oxygen and the recapture system 404 directs at least some of the production output from the oxygen concentrator 402 to the station 410. As the aircraft climbs further or additional demand is required, such as via the station 412, the recapture system 404 can redirect up to 100% of the oxygen concentrator output to the station 410 or station 412. If the volume of oxygen production by the oxygen concentrator cannot meet the demand of the station 410 or 412, the recapture system 404 can supplement the real-time production output of the oxygen concentrator 402 with previously stored and recaptured oxygen from the oxygen reservoir 406.

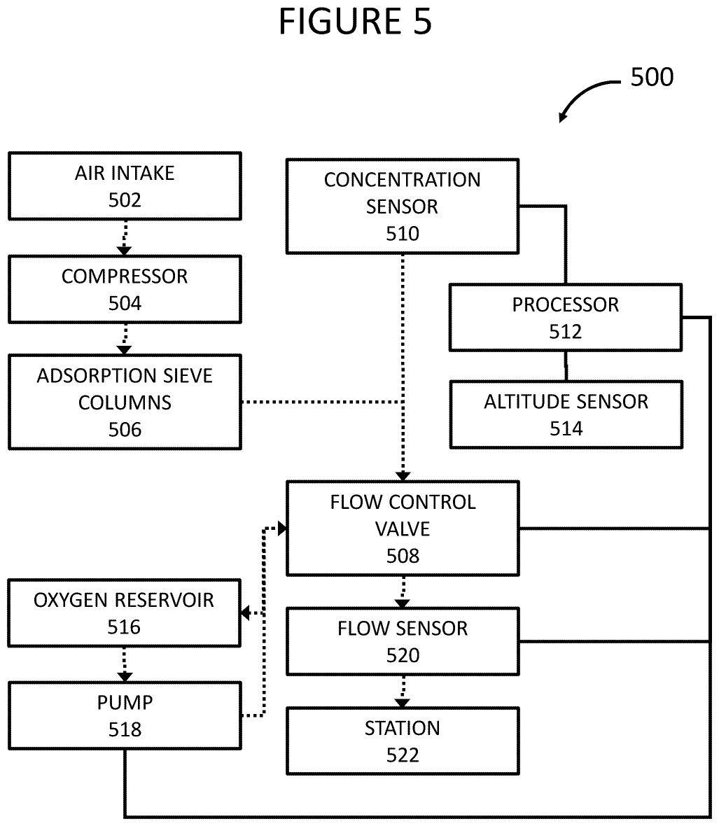

FIG. 5 is a system diagram of a hybrid oxygen delivery system, in accordance with an embodiment of the invention. In one embodiment, an oxygen concentrator system 500 includes, but is not limited to, an oxygen concentrator including an air intake 502, a compressor 504, and adsorption sieve column 506 configured to generate oxygen from ambient air; an oxygen reservoir 516 configured to store the oxygen in reserve when excess oxygen is generated; a recapture system including a flow control valve 508 and a pump 518 configured to source oxygen from the oxygen reservoir 516 and the concentrator 510 when a shortage of oxygen is generated; plumbing (depicted as a dashed line) that fluidly connects the oxygen concentrator 502, 504, 506, the oxygen reservoir 516, and the recapture system 508, 518; and a processor 512 configured to control the recapture system 508, 518 based on one or more parameters to deliver oxygen to one or more stations 522. In one embodiment, the system 500 includes, but is not limited to, a concentration sensor 510, an altitude sensor 514, or a flow sensor 520.

In one embodiment, the oxygen reservoir 516 can include a flexible and inflatable oxygen pillow or bag. The oxygen pillow or bag is formed from flexible non-flammable or fire-resistant material such as PVC and includes a port for filling and discharging oxygen. The oxygen pillow or bag inflates and deflates via the port and can include a pressure safety relief valve. Many sizes are possible, but usable sizes include 35 L, 4 0 L, and 50 L. A plurality of oxygen pillows or bags can be plumbed in parallel or in series to increase a total volume. The oxygen pillow or bag can be incorporated into aircraft voids, such as within the wings, tail cone, or other parts of the fuselage with custom shapes and sizes to fit within space constraints.

The pump 518 is configured to repressurize oxygen stored in the oxygen reservoir 516. The oxygen stored in the oxygen reservoir 516 can of relatively low pressure, which is insufficient to travel through the flow control valve 5108 to the station 522. Accordingly, the processor 512 can actuate the pump 518 to increase the pressure of oxygen output from the oxygen reservoir 516. The pressure gains of the pump 518 can range from approximately 5 PSI to 50 PSI or more. The processor 512 can operate the pump 518 at rates that depend upon altitude and physiological parameters, to supplement the concentrator output and increase the overall total oxygen flow output of the system 500. The processor 512 can disengage the pump 518 when the oxygen supply from the concentrator output is sufficient and redirect any excess concentrator output oxygen to the oxygen reservoir 516 to refill oxygen reserves. In this manner, the system 500 acts as a capacitor for oxygen, storing oxygen in reserves until it is needed and then replenishing the reserves when excess supply is available. During use in an aircraft, this supports higher altitude unpressurized flights for a limited time with a smaller oxygen concentrator that alone would not produce sufficient oxygen flow.

The processor 512 is configured to actuate a flow control valve 508 based on concentration, altitude, physiological demand, or flow. The flow control valve 508 is designed to regulate the flow rate and pressure of gases. These valves operate by adjusting the size of the flow passage, allowing precise control over the gas flow. Common types include ball valves, gate valves, globe valves, and needle valves, each suited for different applications and levels of control. For instance, ball valves provide quick shut-off capabilities, while needle valves offer fine adjustments for precise flow control. Various valves are possible including proportional valves and binary valves which serve different purposes in fluid control systems. Proportional valves provide variable control over the flow rate or pressure of a fluid, allowing for precise adjustments based on the input signal. This makes them ideal for applications requiring fine-tuned control where varying flow rates are necessary. In contrast, binary valves, also known as on/off or directional control valves, operate in a simple open or closed state, providing a straightforward way to start or stop fluid flow. These valves are suitable for applications where only full flow or no flow is needed, such as in basic fluid control systems. The key difference lies in the level of control precision: proportional valves offer continuous modulation, while binary valves provide a binary response. Flow control with a solenoid valve can be achieved by using the valve's electromechanical mechanism to regulate the flow of a liquid or gas. When an electric current is applied to the solenoid coil, it generates a magnetic field that moves a plunger within the valve. This movement either opens or closes the valve, allowing or restricting the flow of the fluid. By varying the duration and frequency of the electrical signal, the solenoid valve can precisely control the flow rate. This makes solenoid valves ideal for applications requiring automated and remote flow control. Their ability to provide quick and accurate adjustments ensures efficient and reliable fluid management.

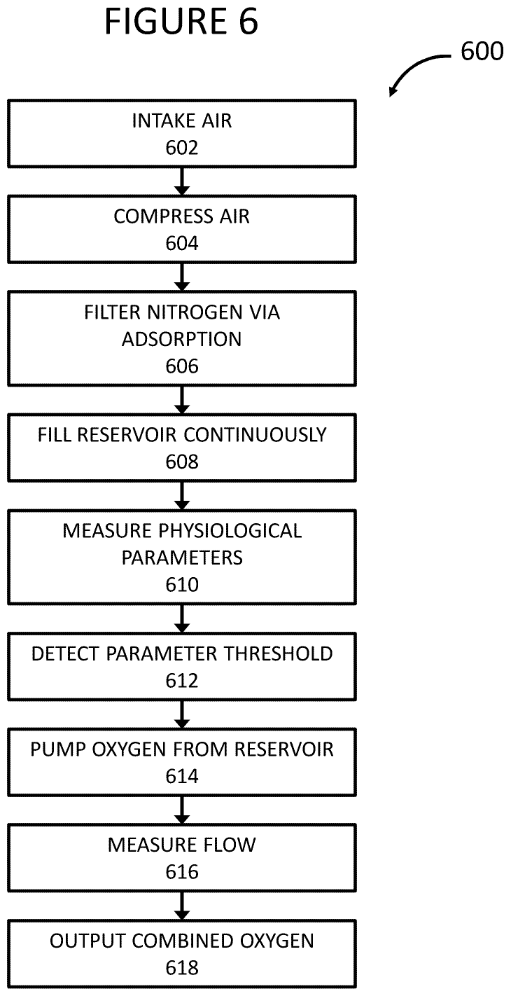

FIG. 6 is a process diagram of a hybrid oxygen delivery system, in accordance with an embodiment of the invention.

In one embodiment, a process 600 for recapturing concentrated oxygen flow includes, but is not limited to, obtaining intake ambient air at 602; compressing the ambient intake air at 604; filtering nitrogen from the ambient intake air to concentrate oxygen using pressure swing adsorption at 606; filling a reservoir with the oxygen continuously at 608; measuring one or more physiological parameters at 610; detecting that the one or more physiological parameters has crossed one or more thresholds at 612; pumping oxygen from the reservoir at a rate that is based on the one or more physiological parameters at 614; measuring a flow of the oxygen at 616; and outputting the oxygen to one or more breathing stations at 618. The process 600 can implement any of the embodiments disclosed herein with respect to various systems.

High altitude hypoxia occurs when the body is exposed to low oxygen levels at high elevations, typically above 2,500 meters (8,200 feet). As altitude increases, atmospheric pressure decreases, leading to a lower partial pressure of oxygen. This reduction in available oxygen can cause symptoms such as shortness of breath, dizziness, fatigue, and impaired cognitive function. The body responds by increasing breathing rate (hyperventilation) and producing more red blood cells to enhance oxygen transport. However, prolonged exposure to high altitude without proper acclimatization can lead to serious conditions like acute mountain sickness (AMS), high-altitude pulmonary edema (HAPE), and high-altitude cerebral edema (HACE). Proper acclimatization and, in some cases, supplemental oxygen are essential to mitigate the effects of high-altitude hypoxia.

Supplemental oxygen works to reduce high altitude hypoxia by increasing the amount of oxygen available to the body, compensating for the lower oxygen levels in the atmosphere at high elevations. When individuals ascend to high altitudes, the reduced atmospheric pressure leads to a decrease in the partial pressure of oxygen, making it harder for the body to obtain sufficient oxygen from the air. By providing a concentrated source of oxygen, supplemental oxygen therapy ensures that the body's tissues receive adequate oxygen, preventing the onset of hypoxia and its associated symptoms such as shortness of breath, dizziness, and cognitive impairment.

The Federal Aviation Administration (FAA) and other aviation bodies recommend specific flow rates for supplemental oxygen to ensure safety at high altitudes. For cabin pressure altitudes up to 40,000 feet, the FAA prescribes minimum mass flow rates to maintain adequate oxygen levels for both crew and passengers. The flow rates vary depending on the altitude and the specific requirements of the aircraft and its occupants. For example, at altitudes above 25,000 feet, higher flow rates are necessary to compensate for the reduced atmospheric pressure and ensure sufficient oxygenation. One rule of thumb is approximately 1 LPM of effective flow per 10 k MSL per station for preventing hypoxia and maintaining cognitive and physical performance during flight.

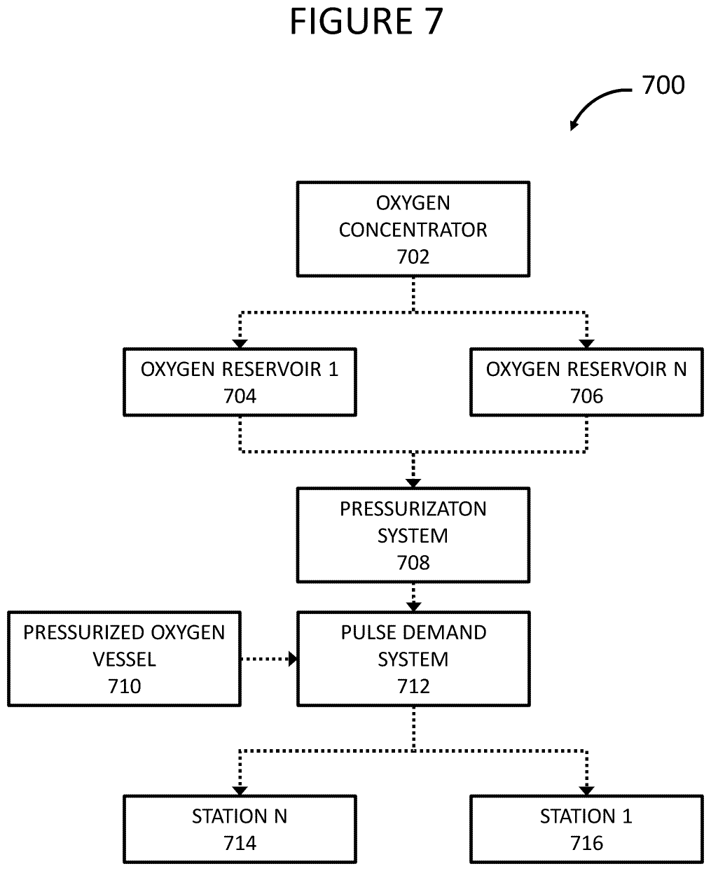

FIG. 7 is a system diagram of a hybrid oxygen delivery system, in accordance with an embodiment of the invention. In one embodiment, system 700 includes, but is not limited to, an oxygen concentrator 702, an oxygen reservoir 704 and optionally one or more additional oxygen reservoirs 706, a pressurization system 708, a pressurized oxygen vessel 710, a pulse demand system 712, station 716 and optionally one or more additional stations 714.

The pulse demand system 712, also known as a pulse dose system, delivers oxygen only when the user inhales, making it more efficient than continuous flow systems. This system uses mass flow or pressure sensors to detect the beginning of an inhalation and then release a precise “pulse” or “bolus” of oxygen. This targeted delivery ensures that oxygen is provided exactly when it is needed, reducing waste and conserving oxygen supply. The amount of oxygen delivered per pulse can be adjusted based on the user's needs.

The pulse of oxygen from the pulse demand system 712 requires pressurization to function and could draw that pressurization from the oxygen concentrator 702 itself. However, relying directly on the pressure from the oxygen concentrator 702 exposes the oxygen concentrator 702 to pressurization drops at each pulse of the pulse demand system 712. If the pressure drop is too significant, the Zeolite of the oxygen concentrator 702 is unable to effectively extract nitrogen, which results in a decrease in oxygen concentration output. Likewise, if the pulse demand system 712 pulses at a slow rate, such as during slow respiration or non-respiration periods, the backup of pressure on the oxygen concentrator 702 can result in excessive heat and wear on the associated compressor pump. Accordingly, the system 700 relies instead on an oxygen reservoir 704 that stores the concentrated oxygen output from the oxygen concentrator 702 at a relatively low pressure. To meet the pressurization needs of the pulse demand system 712, a re-pressurization system 708 is provided that pressurizes a volume of oxygen from the oxygen reservoir 704 in an amount sufficient to serve at least one pulse dosage of the pulse demand system 712. With this embodiment, the oxygen concentrator experiences no loss of oxygen concentration and no increase in temperature due to rhythmic pulsing from the pulse demand system 712.

The optional inclusion of the pressurized oxygen vessel 710 connects to the pulse demand system 712 in parallel with the pressurization system 708. The pulse demand system 712 sources oxygen from the pressurized vessel 710 to supplement or replace the oxygen sourced from the pressurization system 708 and the oxygen reservoir 704 in response to certain threshold events. The threshold events can include insufficient oxygen supply in the oxygen reservoir 704, failure of the oxygen concentrator 702 or pressurization system 708, or loss of alternator or battery power. Additional threshold events can include high altitude flight, low blood oxygen level, high heart rate, low oxygen reservoir volume, the more stations becoming active, or a user request. The pressurized oxygen vessel also serves as a backup with continuous flow oxygen being available independent of the pulse demand system 712 with plumbing lines in parallel to the station 716.

The system 700 therefore enables higher altitude flight with more stations due to recapture of oxygen concentrated at higher volume of oxygen flow output. This is accomplished with a smaller oxygen concentrator with lower power and reduced size as compared to that which would be required without the system 700. Upon exhausting any reservoir, the pressurized vessel 710 is available to supplement or replace oxygen flow of the oxygen concentrator. The pressurized vessel 710 can be smaller (e.g. 47 L or 152 L), with its use being limited to times when the oxygen concentrator 702 and oxygen reservoir 704 become inadequate.

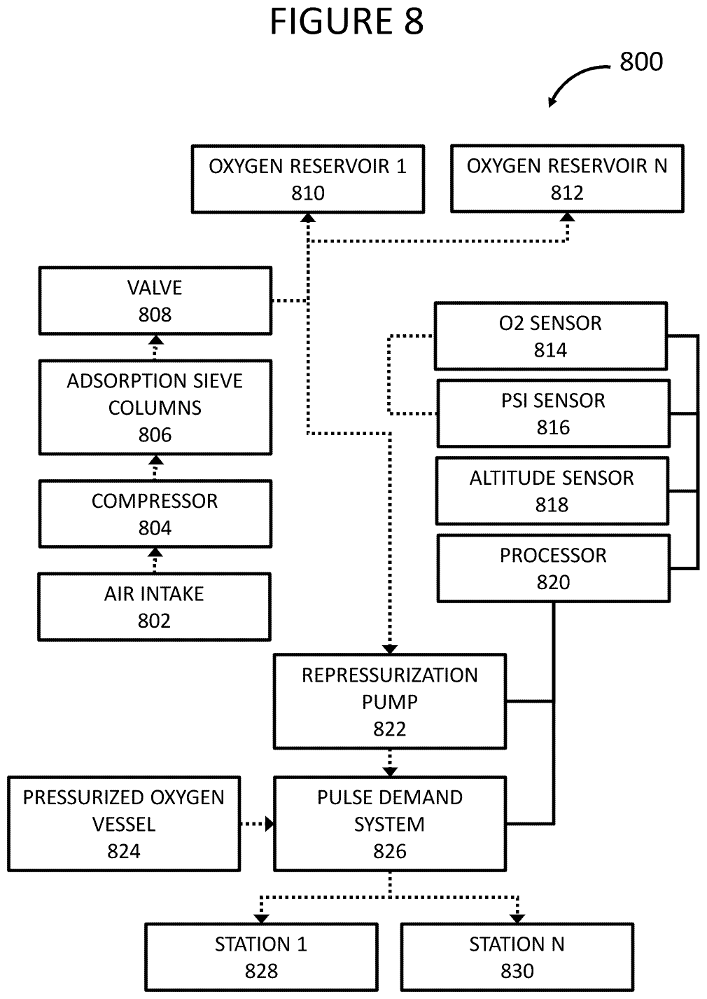

FIG. 8 is a system diagram of a hybrid oxygen delivery system, in accordance with an embodiment of the invention. A hybrid pulse demand oxygen system 800 includes, but is not limited to, an oxygen concentrator with an air intake 802, compressor 804, adsorption sieve column 806, and valve 808 configured to generate oxygen at a first rate; an oxygen reservoir 810 and optionally one or more additional oxygen reservoirs 812 configured to store the oxygen generated at the first rate; an oxygen pressure vessel 824 configured to store pressurized reserve oxygen; a re-pressurization pump 822 configured to increase pressure of the oxygen stored in the oxygen reservoir 810; a pulse demand system 826 configured to deliver oxygen at a variable rate based on one or more parameters obtained from the oxygen sensor 814, pressure sensor 816, physiological sensor (e.g. AITHRE ILLYRIAN), or altitude sensor 818; plumbing (depicted with dashed lines) configured to fluidly connect the oxygen concentrator valve 808, the oxygen reservoir 810, the oxygen pressure vessel 824, the re-pressurization pump 822, and the pulse demand system 826; and a processor 820 configured to variably source oxygen from the re-pressurization pump 822 and the oxygen pressure vessel 824 based on a volume of oxygen available in the oxygen reservoir 810. The oxygen reservoir 810 can include a flexible inflatable oxygen pillow or bag. The pulse demand system 826 includes a breathing detection circuit; and a valve.

The O2 sensor 814 operates by measuring the proportion of oxygen in a gas using various technologies. Common types include electrochemical, zirconia, and optical sensors. Electrochemical sensors generate an electrical current through a chemical reaction with oxygen, providing a proportional measurement of oxygen concentration. Zirconia sensors use a ceramic element that produces a voltage when exposed to oxygen, while optical sensors detect changes in light intensity caused by oxygen presence. These sensors ensure accurate and reliable oxygen measurement.

The oxygen gas pressure sensor 816 measures the pressure exerted by a gas and converts it into an electrical signal that can be processed or displayed. It can operate based on various principles, such as the piezoresistive effect, piezoelectric effect, or capacitive sensing. In piezoresistive sensors, a diaphragm deforms under pressure, causing a change in electrical resistance that is measured and correlated with the pressure applied. Piezoelectric sensors generate an electrical charge when pressure is applied to a piezoelectric material, such as quartz. Capacitive sensors measure changes in capacitance caused by the deformation of a diaphragm under pressure. These sensors provide accurate and reliable pressure measurements.

The altitude sensor 818 measures altitude by detecting changes in atmospheric pressure. A solid-state pressure sensor operates by detecting changes in pressure and converting them into an electrical signal. These sensors typically use microelectromechanical systems (MEMS) technology, which includes a diaphragm that deforms under pressure. In piezoresistive sensors, this deformation changes the resistance of embedded resistors, while in capacitive sensors, it alters the capacitance between electrodes. The deformation of the diaphragm due to pressure changes is converted into an electrical signal, which is then processed by an integrated circuit to provide a digital output. This digital output represents the pressure measurement, allowing for accurate and reliable pressure altitude calculations. A temperature sensor can be combined with the altitude sensor to provide an estimated density altitude for non-standard temperatures.

The physiological sensor can include the AITHRE ILLYRIAN or another wearable physiological sensor. The sensor includes a silicone pad and a built-in pressure altimeter. oximeter, heart rate and variability monitor, movement detector, or carbon monoxide sensing. This wearable sensor provides optional and controllable haptic feedback for a variety of use cases, including depressurization, heart rate surges, low blood oxygen, absence of head movement, high cabin temperature, and elevated carbon monoxide. The wearable sensor is usable in a variety of configurations for headband or under the carcup oximeter placements. The haptic feedback provides a subtle haptic vibration that can be felt and heard. This new modality provides information effectively, yet unobtrusively. The haptic vibrator settings and controls can be setup with a simple pattern to alert the wearer to safety issues. For example, 1 Haptic Vibration=Cabin Temperature or Miscellaneous; 2 Haptic Vibrations=Heart Rate; 3 Haptic Vibrations=SPO2 or Carbon Monoxide: 4 Haptic Vibrations=Cabin Altitude; and 5 Haptic Vibrations=Head Movement. The physiological sensor is usable to provide constant, always-on pulse oximeter readings using a thin sensor pad worn near the ear or on the forehead and then broadcast wirelessly. Additionally, the wearable sensor features continuous heart rate variability and heartbeat monitoring, with continuous output and trend plotting for some key variables.

The processor 820 can use outputs of the oxygen sensor 814, the pressure sensor 816, the altitude sensor 818, and the wearable physiological sensor to control the re-pressurization pump 822. The pulse demand system 826 is reliant on the re-pressurization pump 822 in system 800 to produce oxygen at sufficient pressure to dose to the station 828. The pulse demand system 826 dynamically changes the oxygen dose volume based on parameters such as altitude, blood oxygen levels, respiration rate, and the number of additional active stations 830. Thus, the frequency and duration of operation of the re-pressurization pump 822 can be adjusted dynamically to meet the service needs of the pulse demand system 826. For example, the re-pressurization pump can compress a larger volume of oxygen to a higher pressure in response to an increase in altitude or a decrease in blood oxygen, resulting in more oxygen being available to the pulse demand system. Alternatively, the re-pressurization pump 822 can decrease the duration of actuation to reduce the electrical demand if the O2 sensor 814 or the pressure sensor 816 indicate that the oxygen reservoir 810 is low or empty. This functionality improves the efficiency of the system 800 so that it can be usable with lower alternator capacity and electrical load.

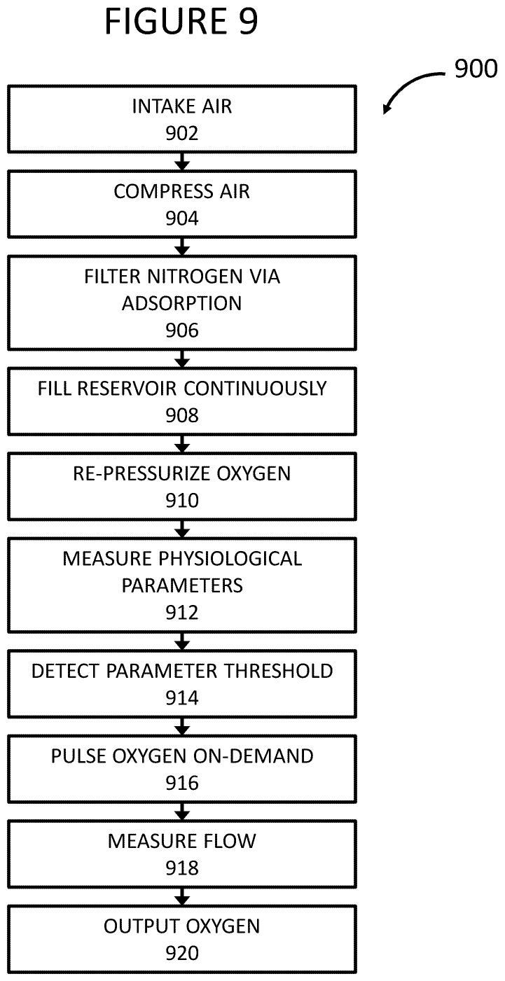

FIG. 9 is a process diagram of a hybrid oxygen delivery system, in accordance with an embodiment of the invention. In one embodiment, a process 900 for pulsing concentrated oxygen includes, but is not limited to, obtaining intake ambient air at 902; compressing the ambient intake air at 904; filtering nitrogen from the ambient intake air to concentrate oxygen using pressure swing adsorption at 906; filling a reservoir with the oxygen continuously at 908; re-pressurizing a portion of the oxygen in the reservoir to create a standby charge for at least one pulse dosage at 910; measuring one or more physiological parameters at 912; detecting that the one or more physiological parameters has crossed one or more thresholds at 914; pulsing oxygen using the standby charge in response to detected breathing in an amount that is based on the one or more physiological parameters at 916; measuring a flow of the oxygen at 918; and outputting the oxygen to one or more breathing stations at 920. The process 900 can implement any of the embodiments disclosed herein with respect to various systems.

While preferred and alternate embodiments of the invention have been illustrated and described, as noted above, many changes can be made without departing from the spirit and scope of the invention. Accordingly, the scope of the invention is not limited by the disclosure of these preferred and alternate embodiments. Instead, the invention should be determined entirely by reference to the claims that follow.

1. A hybrid oxygen delivery system comprising:
a pressurized oxygen vessel configured to store a first source of oxygen under pressure;
an oxygen concentrator configured to generate a second source of oxygen from ambient air;
a flow control valve configured to combine the first source of oxygen with the second source of oxygen for delivery to one or more stations;
plumbing that fluidly connects the pressurized oxygen vessel and the oxygen concentrator to the flow control valve; and
a processor configured to actuate the flow control valve based on one or more parameters to deliver oxygen to one or more stations.

2. The hybrid oxygen delivery system of claim 1, wherein the oxygen concentrator configured to generate a second source of oxygen from ambient air comprises:
an oxygen concentrator including an air intake, a compressor, and an adsorption sieve column.

3. The hybrid oxygen delivery system of claim 1, further comprising:
an oxygen concentration sensor, an altitude sensor, or a flow sensor.

4. The hybrid oxygen delivery system of claim 1, wherein the processor configured to actuate the flow control valve based on one or more parameters comprises:
a processor configured to actuate the flow control valve based on one or more parameters including concentration, altitude, physiological demand, or flow.

5. An oxygen concentrator system comprising:
an oxygen concentrator configured to generate oxygen from ambient air;
an oxygen reservoir configured to store the oxygen in reserve when excess oxygen is generated;
a recapture system configured to source oxygen from the oxygen reservoir and the concentrator when a shortage of oxygen is generated;
plumbing that fluidly connects the oxygen concentrator, the oxygen reservoir, and the recapture system; and
a processor configured to control the recapture system based on one or more parameters to deliver oxygen to one or more stations.

6. The oxygen concentrator system of claim 5, wherein the oxygen concentrator configured to generate oxygen from ambient air comprises:
an oxygen concentrator including an air intake, a compressor, and an adsorption sieve column.

7. The oxygen concentrator system of claim 5, further comprising:
a concentration sensor, an altitude sensor, or a flow sensor.

8. The oxygen concentrator system of claim 5, wherein the recapture system configured to source oxygen from the oxygen reservoir and the concentrator when a shortage of oxygen is generated comprises:
a pump configured to repressurize oxygen stored in the oxygen reservoir; and
a flow control valve.

5. he oxygen concentrator system of claim 5, wherein the processor configured to control the recapture system based on one or more parameters to deliver oxygen to one or more stations comprises:
a processor configured to actuate a flow control valve based on concentration, altitude, physiological demand, or flow.

10. The oxygen concentrator system of claim 5, wherein the oxygen reservoir configured to store the oxygen in reserve when excess oxygen is generated comprises:
an oxygen reservoir including a flexible and inflatable oxygen pillow.

11. A hybrid pulse demand oxygen system comprising:
an oxygen concentrator configured to generate oxygen at a first rate;
an oxygen reservoir configured to store the oxygen generated at the first rate;
an oxygen pressure vessel configured to store pressurized reserve oxygen;
a re-pressurization pump configured to increase pressure of the oxygen stored in the oxygen reservoir;
a pulse demand system configured to deliver oxygen at a variable rate based on one or more parameters;
plumbing configured to fluidly connect the oxygen concentrator, the oxygen reservoir, the oxygen pressure vessel, the re-pressurization pump, and the pulse demand system; and
a processor configured to variably source oxygen from the re-pressurization pump and the oxygen pressure vessel based on a volume of oxygen available in the oxygen reservoir.

12. The hybrid pulse demand system of claim 11, wherein the oxygen concentrator comprises:
an air intake, a compressor, an adsorption sieve column; and a valve.

13. The hybrid pulse demand system of claim 11, wherein the oxygen reservoir comprises:
a flexible inflatable oxygen pillow.

14. The hybrid pulse demand system of claim 11, further comprising:
an oxygen sensor, a pressure sensor, a physiological sensor, or an altitude sensor.

15. The hybrid pulse demand system of claim 11, wherein the pulse demand system comprises:
a breathing detection circuit; and
a valve.

16. A process for producing hybrid oxygen flow comprising:
obtaining intake ambient air;
compressing the ambient intake air;
filtering nitrogen from the ambient intake air to concentrate oxygen using pressure swing adsorption;
measuring one or more physiological parameters;
detecting that the one or more physiological parameters has crossed one or more thresholds;
supplementing the oxygen with reserve oxygen in response to detecting that the one or more physiological parameters has crossed the one or more thresholds;
measuring a flow of the oxygen; and
outputting the oxygen to one or more breathing stations.

17. A process for recapturing concentrated oxygen flow comprising:
obtaining intake ambient air;
compressing the ambient intake air;
filtering nitrogen from the ambient intake air to concentrate oxygen using pressure swing adsorption;
filling a reservoir with the oxygen continuously;
measuring one or more physiological parameters;
detecting that the one or more physiological parameters has crossed one or more thresholds;
pumping oxygen from the reservoir at a rate that is based on the one or more physiological parameters;
measuring a flow of the oxygen; and
outputting the oxygen to one or more breathing stations.

18. A process for pulsing concentrated oxygen comprising:
obtaining intake ambient air;
compressing the ambient intake air;
filtering nitrogen from the ambient intake air to concentrate oxygen using pressure swing adsorption;
filling a reservoir with the oxygen continuously;
re-pressurizing a portion of the oxygen in the reservoir to create a standby charge for at least one pulse dosage;
measuring one or more physiological parameters;
detecting that the one or more physiological parameters has crossed one or more thresholds;
pulsing oxygen using the standby charge in response to detected breathing in an amount that is based on the one or more physiological parameters;
measuring a flow of the oxygen; and
outputting the oxygen to one or more breathing stations.
